(19) United States
(12) Patent Application Publication
(10) Pub. No.: US 2014/0155652 A1
(43) Pub. Date: Jun. 5, 2014

(54) METHOD OF PRODUCING METHIONINE

(71) Applicant: SUMITOMO CHEMICAL COMPANY, LIMITED, Tokyo (JP)

(72) Inventors: Naoya YAMASHIRO, Niihama-shi (JP); Junichi NISHIDA, Niihama-shi (JP); Yoshiyuki KOIZUMI, Niihama-shi (JP)

(73) Assignee: SUMITOMO CHEMICAL COMPANY, LIMITED, Tokyo (JP)

(21) Appl. No.: 14/082,542

(22) Filed: Nov. 18, 2013

(30) Foreign Application Priority Data
Dec. 4, 2012 (JP) 2012-265054

(51) Int. Cl. C07C 319/12

(52) U.S. Cl. CPC C07C 319/12
USPC 562/559

(57) ABSTRACT

The invention provides the following method capable of producing methionine in a shorter time by making rapid progress of the hydrolysis of 5-(2-methylmercaptoethyl)hydantoin from an aqueous 5-(2-methylmercaptoethyl)hydantoin solution containing ammonia component. A method of producing methionine is the method comprising hydrolyzing 5-(2-methylmercaptoethyl)hydantoin in an aqueous 5-(2-methylmercaptoethyl)hydantoin solution containing ammonia component, wherein the hydrolysis is performed after the ammonia component is removed from the aqueous solution.

### BACKGROUND OF THE INVENTION

1. Field of the Invention

The present application is filed, claiming the Paris Convention priority based on Japanese Patent Application No. 2012-265054 (filed on Dec. 4, 2012), and the entire content of which is incorporated herein by reference.

The present invention relates to a method of producing methionine.

2. Description of the Related Art

Methionine is useful as feed additive. As a method for producing such a methionine, a method in which 5-(2-methylmercaptoethyl)hydantoin is heated in the presence of an alkali component in water to hydrolyze is well known (JP-A-2007-314507).

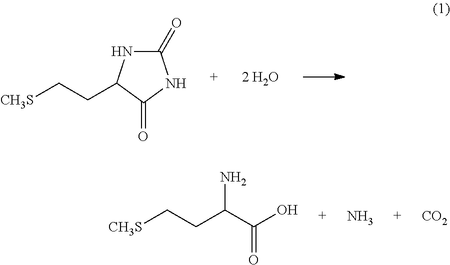

5-(2-methylmercaptoethyl)hydantoin which is a raw material can be obtained by, for example, a method in which 3-methylmercaptopropionaldehydecyanhydrin is reacted with a carbonic acid and an ammonia (JP-A-5-286926).

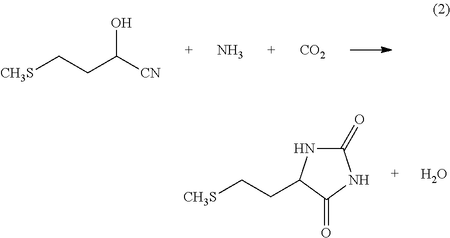

In this reaction, an ammonia is usually used in an excess amount and therefore, an unreacted ammonia is contained in the aqueous 5-(2-methylmercaptoethyl)hydantoin solution obtained as the reaction solution after the reaction is completed. The aqueous solution is usually used for hydrolysis.

A rapid progress of the hydrolysis of 5-(2-methylmercaptoethyl)hydantoin is desirable from the viewpoint of production efficiency of methionine.

### SUMMARY OF THE INVENTION

The inventors of the present invention have made earnest studies for making more rapid progress of the hydrolysis of 5-(2-methylmercaptoethyl)hydantoin, and found that ammonia component contained in an aqueous 5-(2-methylmercaptoethyl)hydantoin solution retards the progress of the hydrolysis of 5-(2-methylmercaptoethyl)hydantoin. The inventors of the present invention focused on this find and found that the hydrolysis rapidly proceed and methionine can be produced in a relatively short time by hydrolyzing 5-(2-methylmercaptoethyl)hydantoin after removing ammonia component contained in the aqueous 5-(2-methylmercaptoethyl)hydantoin solution.

Accordingly, the present invention is as follows:

[1] A method of producing methionine, the method comprising hydrolyzing 5-(2-methylmercaptoethyl)hydantoin in an aqueous 5-(2-methylmercaptoethyl)hydantoin solution containing ammonia component, wherein the hydrolysis is performed after the ammonia component is removed from the aqueous solution.

[2] The production method according to above [1], wherein the aqueous 5-(2-methylmercaptoethyl)hydantoin solution containing ammonia component is a reaction solution obtained after 3-methylmercaptopropionaldehydecyanhydrin is reacted with a carbonic acid component and an excess amount of an ammonia component in the presence of water.

[3] The production method according to above [1] or [2], wherein the ammonia component is removed by blowing inert gas into the aqueous 5-(2-methylmercaptoethyl)hydantoin solution containing ammonia component.

According to the production method of the present invention, methionine can be produced in a shorter time from an aqueous 5-(2-methylmercaptoethyl)hydantoin solution containing ammonia component.

### DETAILED DESCRIPTION OF THE PREFERRED EMBODIMENTS

(Aqueous 5-(2-methylmercaptoethyl)hydantoin solution)

The content of 5-(2-methylmercaptoethyl)hydantoin contained in the aqueous 5-(2-methylmercaptoethyl)hydantoin solution used in the production method of the present invention is usually from 1 to 50% by weight and preferably from 10 to 20% by weight.

This aqueous solution contains ammonia components. Examples of the ammonia components include ammonium ion and ammonia. The content of these ammonia components in the solution is usually from 2 to 7% by weight and preferably from 3 to 6% by weight calculated as ammonia and usually from 1 to 4 mol and preferably from 2 to 3 mol calculated as ammonia per 1 mol of 5-(2-methylmercaptoethyl)hydantoin.

This aqueous solution may contain, for example, 3-methylmercaptopropionaldehydecyanhydrin or carbonic acid component besides 5-(2-methylmercaptoethyl)hydantoin and ammonia component. Examples of the carbonic acid components include carbonic acid ion and hydrogen carbonate ion and the content of these carbonic acid component is usually from 2 to 7% by weight.

The aqueous 5-(2-methylmercaptoethyl)hydantoin solution may be obtained, for example, by a method in which 3-methylmercaptopropionaldehydecyanhydrin is reacted with a carbonic acid component and an excess amount of ammonia component in the presence of water.

The used amount of water is usually from 3 to 4 times of weight to the weight of 3-methylmercaptopropionaldehydecyanhydrin.

As the carbonic acid component, for example, ammonium carbonate is used. The amount of the carbonic acid component is usually from 1 to 5 mol and preferably from 1.5 to 3 mol calculated as carbon dioxide per 1 mol of 3-methylmercaptopropionaldehydecyanhydrin.

As the ammonia component, ammonia is used. The ammonia component is used in an excess amount exceeding usually 2 mol and preferably from 3 to 5 mol calculated as ammonia per 1 mol of 3-methylmercaptopropionaldehydecyanhydrin.

Ammonium carbonate may be used as the component of both the carbonic acid component and ammonia component. In this case, the amount of ammonium carbonate is usually from 0.7 to 3 times of weight and preferably from 0.9 to 2 times of weight to the weight of 3-methylmercaptopropionaldehydecyanhydrin.

The reaction may be performed, for example, with dissolving the carbonic acid component and ammonia component in water and blending the mixture with 3-methylmercaptopropionaldehydecyanhydrin followed by heating. The reaction temperature is usually from 50° C. to 90° C. and the reaction time is usually from 0.5 to 6 hr.

An aqueous 5-(2-methylmercaptoethyl)hydantoin solution is obtained as the reaction solution after the completion of the reaction. This aqueous solution is subjected to the subsequent hydrolysis without any aftertreatment. This aqueous solution contains unreacted ammonia component.

### (Removal of Ammonia Components)

In the production method of the present invention, hydrolysis reaction is performed after ammonia component is removed from an aqueous 5-(2-methylmercaptoethyl)hydantoin solution. As a method of removing ammonia components, a method of blowing inert gas into the aqueous 5-(2-methylmercaptoethyl)hydantoin solution is usually adopted. The inert gas is, for example, blown into the aqueous solution from a pipe with one end inserted into the aqueous solution. The inert gas is preferably blown in a dispersion state into the aqueous solution, for example, through a bubble generator which is attached to the end of a pipe and blows the inert gas as air cells into the aqueous solution, and more precisely, a bubble generator made of porous ceramic or silicone resin. Also, the inert gas may be blown as air cells into the aqueous solution from a sparger.

For example, nitrogen gas or air is used as the inert gas and the blown amount of the inert gas is usually from 5 to 200 kg/hr, preferably from 10 to 100 kg/hr, and more preferably from 20 to 60 kg/hr per 1000 kg of the aqueous solution.

When the inert gas is blown, the temperature of the aqueous 5-(2-methylmercaptoethyl)hydantoin solution is usually from 30° C. to 70° C. and preferably from 40° C. to 60° C. and the solution usually has a hydrogen ion concentration of pH from 9 to 14. Also, the time for blowing inert gas is usually from 200 min to 1200 min and preferably from 400 to 800 min. The inert gas is preferably dispersed in the state of fine air cells into the aqueous solution and therefore the blowing is desirably conducted with a sparger or the like.

Ammonia component in the aqueous solution can be removed by blowing inert gas into the aqueous 5-(2-methylmercaptoethyl)hydantoin solution as mentioned above. The removing operation is performed to the extent that the content of ammonia component in the aqueous solution is usually 0.2% by weight or less and ideally 0% by weight calculated as ammonia. This removing operation may be performed to the extent that the content of ammonia component in the aqueous solution is usually 0.05 mol or less and ideally 0 mol calculated as ammonia per 1 mol of 5-(2-methylmercaptoethyl)hydantoin.

(Hydrolysis of 5-(2-methylmercaptoethyl)hydantoin)

In the production method of the present invention, the aqueous 5-(2-methylmercaptoethyl)hydantoin solution from which ammonia components are removed as mentioned above is hydrolyzed. The hydrolysis is performed with adding an alkali component for increasing the hydrogen ion concentration in the aqueous solution to pH 10 or more, followed by heating, as described in JP-A-2007-314507.

Usually, alkali metal carbonates such as potassium carbonate; alkali metal hydrogen carbonates such as potassium hydrogencarbonate; or alkali metal hydroxides such as potassium hydroxide are used as the alkali component. The used amount of the alkali component is usually from 0.5 to 6 mol and preferably from 1 to 3 mol per 1 mol of 5-(2-methylmercaptoethyl)hydantoin.

The hydrolysis is performed by heating the aqueous solution to the temperature of, usually, 100° C. to 220° C., and preferably 150° C. to 200° C. under a pressure of about from 0.5 to 1 MPa (gauge pressure). The reaction time is usually from 10 min to 24 hr and preferably 20 min to 2 hr.

### (Extraction of Methionine)

In order to extract methionine from the hydrolytic solution thus obtained, carbon dioxide is introduced into the reaction solution to carryout crystallization and the obtained slurry is separated into a precipitate and mother liquor by, for example, filtration or decantation to obtain precipitated methionine.

Carbon dioxide is dissolved into the reaction solution by the introduction of carbon dioxide, resulting in precipitating a potassium salt of methionine as free methionine.

Carbon dioxide is preferably introduced under a pressure of usually from 0.1 to 1 MPa and preferably from 0.2 to 0.5 MPa (gauge pressure).

The crystallization temperature is usually from 0 to 50° C. and preferably from 10 to 30° C. While the time until carbon dioxide is saturated in the hydrolytic reaction solution and methionine is completely precipitated may be regarded as a standard time for the crystallization time, the crystallization time is usually 30 min to 24 hr.

Separated methionine may be subjected to washing and pH adjustment, if necessary, and then dried to obtain a product. This drying is preferably performed by heating the above separated methionine to the temperature of from about 50 to 120° C. under slightly reduced pressure and the drying time is usually from 10 min to 24 hr.

### EXAMPLES

The present invention will be explained in more detail by examples, which are not intended to be limiting of the present invention.

### Comparative Example 1

Ammonium carbonate was reacted with 3-methylmercaptopropionaldehydecyanhydrin at 75° C. for 2.5 hr in water to obtain an aqueous 5-(2-methylmercaptoethyl)hydantoin solution containing 15% by weight of 5-(2-methylmercaptoethyl)hydantoin and an ammonia component in an amount of 3.6% by weight calculated as ammonia. This aqueous solution had a hydrogen ion concentration of pH 9.9. The used amount of ammonium carbonate was 1.15 times of weight and the used amount of water was 3.5 times of weight to the amount of 3-methylmercaptopropionaldehydecyanhydrin. An aqueous 50% potassium hydroxide solution (used amount: 80 parts by weight) was added to this aqueous solution (600 parts by weight) at ambient temperature until the hydrogen ion concentration of the solution reached pH 10.5 and the mixture was then heated to 120° C. at a rate of 4° C./min and to 173° C. at a rate of 2° C./min from a temperature exceeding 120° C., followed by a reaction at the same temperature (173° C.). The yield of methionine at 10 minutes after the temperature of the reaction solution reached to the temperature of 173° C. was 31.6% (measured by HPLC).

### Example 1

The aqueous 5-(2-methylmercaptoethyl)hydantoin solution (600 parts by weight) obtained in Comparative Example 1 was heated to the temperature of 50° C. while blowing nitrogen gas in the state of air cells under atmospheric pressure at a flow rate of 25 parts by weight/hr from a soft sponge attached to the end of a pipe and that temperature was kept for 8 hr to remove ammonia component. The content of ammonia component in the aqueous solution after the removing operation was less than 0.1% by weight (lower limit for detection) calculated as ammonia (measured by HPLC). When an aqueous 50% by weight of potassium hydroxide solution (used amount: 150 parts by weight) was added to the obtained aqueous solution at ambient temperature until the hydrogen ion concentration reached to pH 10.5 and the resulting solution was heated and reacted in the same manner as in Comparative Example 1, the yield of methionine at 10 minutes after the temperature of the reaction solution reached to the temperature of 173° C. was 53.6% (measured by HPLC).

According to the production method of the present invention, methionine can be produced in a shorter time from an aqueous 5-(2-methylmercaptoethyl)hydantoin solution containing ammonia component.

1. A method of producing methionine, the method comprising hydrolyzing 5-(2-methylmercaptoethyl)hydantoin in an aqueous 5-(2-methylmercaptoethyl)hydantoin solution containing ammonia component, wherein the hydrolysis is performed after the ammonia component is removed from the aqueous solution.

2. The production method according to claim 1, wherein the aqueous 5-(2-methylmercaptoethyl)hydantoin solution containing ammonia component is a reaction solution obtained after 3-methylmercaptopropionaldehydecyanhydrin is reacted with a carbonic acid component and an excess amount of an ammonia component in the presence of water.

3. The production method according to claim 1, wherein the ammonia component is removed by blowing inert gas into the aqueous 5-(2-methylmercaptoethyl)hydantoin solution containing ammonia component.
